(19) United States
(12) Patent Application Publication
(10) Pub. No.: US 2026/0308514 A1
(43) Pub. Date: Oct. 8, 2026

(54) COMPOSITION FOR CULTIVATION OF STEM CELLS INCLUDING SUBSTRATE WITH CONTROLLED MECHANICAL STRENGTH AND USE THEREOF

(71) Applicant: AJOU UNIVERSITY INDUSTRY-ACADEMIC COOPERATION FOUNDATION, Suwon-si, Gyeonggi-do (KR)

(72) Inventors: Byung Gon KIM, Seoul (KR); Hee Hwan PARK, Suwon-si (KR)

(73) Assignee: AJOU UNIVERSITY INDUSTRY-ACADEMIC COOPERATION FOUNDATION, Suwon-si, Gyeonggi-do (KR)

(21) Appl. No.: 18/998,244

(22) PCT Filed: Jul. 5, 2023
(86) PCT No.: PCT/KR2023/009502

§ 371 Date: Jan. 24, 2025

(30) Foreign Application Priority Data
Jul. 25, 2022 (KR) 10-2022-0091590

(51) Int. Cl. C12N 5/0797; A61K 35/30

(52) U.S. Cl. CPC C12N 5/0623; A61K 35/30

(57) ABSTRACT

Neural stem cells cultivated in the cultivation composition of the present disclosure, the cultivation composition comprising a substrate with controlled mechanical strength (for example, hydrogel), are excellent in cell adhesion and survival rate after transplantation and can advantageously be utilized as a cell therapy.

### TECHNICAL FIELD

The present disclosure relates to a composition for cultivating stem cells, comprising a substrate with controlled mechanical strength and use thereof, and more specifically to a composition for cultivating stem cells and a scaffold material comprising a substrate with controlled mechanical strength, a method for cultivating stem cells comprising cultivating stem cells with the composition for cultivating stem cells and a cell therapy comprising the scaffold material.

### RELATED ART

The loss of neurological function that accompanies central nervous system damage such as spinal cord injury or stroke occurs due to damage to the neural circuit as a result of the death of nerve cells. Particularly, in the case of spinal cord injury, motor neuron cells in the spinal cord that are involved in body movement die due to the harmful environment and secondary damage within the damaged area. In addition, the axons (hereinafter, referred to as axons) originating from the motor nerve center of the brain are cut due to damage, thereby breaking the neural network connection between the spinal cord and the motor neurons of the brain. Mature central nervous system axons lack the ability to spontaneously regenerate after injury, and many central nervous system patients suffer from permanent motor function impairments such as lower limb paralysis or quadriplegia. In order to prevent or treat these neurological disorders, there is currently no clear method used in clinical practice other than temporary symptom relief therapy using high doses of steroids.

In order to rebuild damaged neural circuits, much research is being conducted on ways to reconstruct damaged neural circuits by transplanting neural stem cells (NSCs) with high differentiation capacity into brain motor neurons into the damaged spinal cord. However, there are limitations such as the death of neural stem cells and insufficient differentiation capacity into nerve cells in a damaged biological environment.

Accordingly, the inventors of the present disclosure have made diligent efforts to develop a reconstruction treatment technology for neural circuits by increasing the transplant survival rate of neural stem cells. As a result, the inventors of the present disclosure have confirmed that the adhesive ability and survival rate of neural stem cells can be controlled by the mechanical strength of a substrate around the cells, and the present disclosure was completed by identifying and using the molecular mechanism therefor.

### SUMMARY

An object of the present disclosure is to provide a composition for cultivating stem cells, comprising a substrate with controlled mechanical strength and use thereof.

In order to achieve the above object, the present disclosure provides a composition for cultivating stem cells, the composition comprising: a substrate having a mechanical strength of 0.2 kPa to 500 kPa.

In the present disclosure, the substrate includes a hydrogel.

In the present disclosure, the hydrogel comprises a natural polymer, a synthetic polymer or a hybrid thereof.

In the present disclosure, the hydrogel further comprises an actin fiber activity regulator or a mechanoreceptor activity regulator.

In the present disclosure, the stem cells include neural stem cells.

In addition, the present disclosure provides a method for cultivating stem cells, the method comprising: cultivating stem cells with the composition.

In addition, the present disclosure provides a scaffold material for cultivating stem cells, the scaffold material comprising: a substrate having a mechanical strength of 0.2 kPa to 500 kPa.

In the present disclosure, the substrate includes a hydrogel.

In the present disclosure, the hydrogel comprises a natural polymer, a synthetic polymer or a hybrid thereof.

In the present disclosure, the hydrogel further comprises an actin fiber activity regulator or a mechanoreceptor activity regulator.

In the present disclosure, the stem cells include neural stem cells.

In addition, the present disclosure provides a cell therapy comprising the scaffold material as an active ingredient.

In the present disclosure, the cell therapy is for treating a nervous system disease.

In the present disclosure, neural stem cells cultivated on a substrate with controlled mechanical strength (e.g., hydrogel) have the advantage of being able to be utilized as a cell therapy, because their cell adhesion ability and survival rate are significantly improved after transplantation.

### BRIEF DESCRIPTION OF THE DRAWINGS

FIG. 1 relates to the results of confirming changes in cell shape of NSC (neural stem/progenitor cells) as mechanical strength increased. FIG. 1A shows a phase/contrast image, and FIG. 1B shows a fluorescence image. In FIG. 1, kPa represents Kilo pascal (a unit representing mechanical strength), and GFP NSC represents NSC (neural stem/progenitor cells) neural stem cells that exhibit green fluorescent protein (GFP) fluorescence.

FIG. 2 relates to quantitative analysis of cell shape changes in neural stem cells as mechanical strength increased.

FIG. 3 relates to the results of confirming the survival rate of neural stem cells according to an increase in mechanical strength. In FIG. 3, the Live/dead cytotoxicity assay kit was used as a reagent, and Calcein AM (green, live cells) and Ethidium homodimer-1 (red, dead cells) are shown.

FIG. 4 relates to quantitative analysis of the survival rate of neural stem cells according to an increase in mechanical strength.

FIG. 5 relates to RT-PCR results confirming cellular receptors that responded to mechanical sensitivity, and brain and lung samples were used as positive controls, and 18S refers to the housekeeping gene.

FIG. 6 relates to changes in spontaneous calcium oscillation (calcium ion concentration change) inside cells according to mechanical sensitivity. Calcium levels inside cells were measured using the Fluo4AM calcium indicator, and spontaneous calcium oscillations of neural stem cells according to mechanical sensitivity were confirmed in real time.

FIG. 7 relates to quantitative analysis of changes in calcium ion concentration inside cells according to mechanical sensitivity. (A) represents a normalized intensity, (B) represents a calcium responsive rate, (C) represents an amplitude, and (D) represents a frequency.

FIG. 8 relates to cell shape changes caused by mechanical stimulation cell receptor inhibitors in response to mechanical sensitivity. GsMtx4 is a well-known mechanosensitive cell receptor inhibitor that responds to mechanical sensitivity.

FIG. 9 relates to quantitative analysis of cell shape changes caused by cell receptor inhibitors in response to mechanical sensitivity.

FIG. 10 relates to changes in cell survival rate caused by cell receptor inhibitors in response to mechanical sensitivity. In FIG. 10, the Live/dead cytotoxicity assay kit was used as a reagent, and Calcein AM (green live cells) and Ethidium homodimer-1 (red dead cells) are shown.

FIG. 11 relates to quantitative analysis of changes in cell survival rate caused by cell receptor inhibitors in response to mechanical sensitivity.

FIG. 12 relates to the results of measuring the elasticity of cells using a high-speed atomic force microscope (kPa (Kilo pascal) refers to a unit representing mechanical strength). FIG. 12A shows the force and separation distance when neural stem cells were cultivated on hydrogel substrates of 12, 25 and 100 kPa, the cells were fixed, and the high-speed atomic force microscope cantilever (probe) approached the cells. FIG. 12B is a quantitative analysis of the final results of cell elasticity by applying and calculating the Sneddon model to the results for FIG. 12A.

FIG. 13 relates to cell elasticity due to differences in the mechanical strength of the substrate (DMSO: control drug, Cyto. D: actin fiber destabilizing drug (cytochalasin D)).

FIG. 14 relates to spontaneous calcium oscillation changes due to differences in the mechanical strengths of the substrates.

FIG. 15 relates to the adhesion ability and cell shape of neural stem cells (Scale: 50 μm (top), 20 μm (bottom)). FIG. 15A to B are the imaging results after siRNA-delivered neural stem cells were cultivated on a hydrogel substrate for 24 hours and the cells were fixed (ascending scale bar: 50 micrometers, descending scale bar: 20 micrometers). FIG. 15C to E are graphs showing quantitative measurements of cell adhesion (C), cell spreading area (D) and cell perimeter (E) induced by Piezo-1 siRNA in neural stem cells cultivated on 0.2 kPa and 25 kPa substrates.

### DETAILED DESCRIPTION OF THE EMBODIMENTS

Hereinafter, the present disclosure will be described in detail.

Unless otherwise defined, all technical and scientific terms used in the present specification have the same meanings as commonly understood by a person skilled in the art to which the present disclosure pertains. In general, the nomenclature used herein is well known and commonly used in the art.

Cell transplantation treatment for spinal cord injury is problematic due to the poor survival rate of transplanted cells. Biomaterial-based scaffolds aid cell engraftment in the harsh microenvironment of the injured spinal cord. In previous studies, the inventors of the present disclosure have demonstrated that the injection of a thermosensitive injectable hydrogel can generate extracellular matrix (ECM) at lesion epicenters, which can develop into cystic cavities. Accordingly, it was expected that the space-filling effect of the hydrogel would be very helpful for graft survival, but a phenomenon was observed in which NSCs delivered as a complex with the hydrogel barely survived transplantation due to frequent transplantation failures.

Through accumulated evidence, the inventors of the present disclosure have confirmed that mechanical stimulation transmitted to the cell membrane can have a significant impact on cell behavior, and accordingly, it was assumed that the mechanical stiffness of a hydrogel may be an important factor in regulating the survival of neural stem cells transplanted into a hydrogel composite.

The inventors of the present disclosure designed an in vitro culture system to cultivate E14 spinal cord-derived neural stem cells on hydrogel substrates with various strengths ranging from 0.2 to 25 kPa. Neural stem cells cultivated on hard substrates had increased spreading area and cellular perimeter, and improved cell adhesion. Additionally, the ratio of living and dying cells significantly increased.

The inventors of the present disclosure confirmed that neural stem cells are activated by mechanical responses and express several mechanosensitive ion channels that induce calcium currents. The degree of calcium oscillation of neural stem cells cultivated in a 25 kPa hydrogel was significantly increased compared to neural stem cells cultivated in a 0.2 kPa hydrogel. On the other hand, the pharmacological inhibition of mechanosensory-sensitive channels using GsMTx4 attenuated the intensity-dependent enhancement of cytoplasmic expansion, adhesive ability and cell survival of neural stem cells. Through the above mechanism, it was confirmed that the adhesion ability and survival rate of neural stem cells can be controlled by the mechanical strength of the matrix around the cells.

Accordingly, the present disclosure relates to a composition for cultivating stem cells, the composition comprising: a substrate having a mechanical strength of 0.2 kPa to 500 kPa.

In the present disclosure, the mechanical strength of the substrate may vary depending on the mechanical strength of spinal cord tissue of humans and animals. The mechanical strength of spinal cord tissue may vary depending on the species of animal. For example, the mechanical strength of human spinal cord tissue is about 100 kPa, and the mechanical strength of porcine spinal cord tissue is about 500 kPa.

In the present disclosure, the term “stem cells” refer to cells that have the ability to self-replicate and differentiate into two or more different types of cells.

In the present disclosure, the substrate includes a hydrogel, but is not limited thereto.

In the present disclosure, the hydrogel comprises a natural polymer, a synthetic polymer or a hybrid thereof. Natural polymers may include, but are not limited to, collagen, elastin, fibrin, silk, matrigel, hyaluronic acid (HA), chitosan, collagen, laminin and gelatin. Synthetic polymers may include poly hydroxyethyl methacrylate (PHEMA), poly hydroxypropyl methacrylate (PHPMA), poly propylene fumarate (PPF), poly vinyl alcohol (PVA), poly ethylene glycol (PEG), poly ethylene glycol monoacrylate (PEGMA), polyacrylamide (PAAM) and the like, but are not limited thereto. Additionally, hybrids may be ingredients that blend or copolymerize natural polymers and synthetic polymers.

In the present disclosure, the hydrogel further comprises an actin fiber activity regulator or a mechanoreceptor activity regulator. Additionally, according to one embodiment of the present disclosure, the actin fiber activity regulator may be cytochalasin D, and the mechanoreceptor activity regulator may be GsMtx-4.

In the present disclosure, the stem cells include neural stem cells, but are not limited thereto.

From another aspect, the present disclosure relates to a method for cultivating stem cells, the method comprising: culturing stem cells with the composition.

From still another aspect, the present disclosure relates to a scaffold material for cultivating stem cells, the scaffold material comprising: a substrate with a mechanical strength of 0.2 kPa to 500 kPa.

In the present disclosure, the term “scaffold” may be referred to as a “support” and is a term used in tissue engineering, and it refers to a structure that uses a combination of cells and various materials to anchor living cells. In addition, bio-scaffold refers to a biological structure, that is, a structure (mold) that removes cells by decellularizing a living organ and leaving only the microstructure and outline of the organ.

In the present disclosure, the substrate includes a hydrogel, but is not limited thereto.

Since the features corresponding to the above-described hydrogel included in the scaffold material can be replaced in the above-mentioned part, the description thereof will be omitted.

In the present disclosure, the stem cells include neural stem cells, but are not limited thereto.

From still another aspect, the present disclosure relates to a cell therapy comprising the scaffold material as an active ingredient.

In the present disclosure, the term “cell therapy” refers to cells and tissues produced through isolation, culture and special manipulation from a subject, and is a medicine used for treatment, diagnosis and prevention purposes, and it refers to a medicine that is used for the purposes of treating, diagnosing and preventing a disease through a series of actions such as proliferating and selecting living autologous or xenogeneic cells in vitro or changing the biological characteristics of cells through other methods to restore the function of cells or tissues.

In the present disclosure, the cell therapy is for treating a nervous system disease, but is not limited thereto. Specifically, the cell therapy may be used for regenerative treatment of traumatic and non-traumatic central nervous system damage diseases accompanied by tissue defects.

In the present disclosure, the cell therapy composition may preferably include 0.01 to 99.9 wt%, and more preferably 0.1 to 99 wt% of the stem cells based on the total weight.

In the present disclosure, the cell therapy composition may further include appropriate carriers, excipients and diluents commonly used in the preparation of cell therapy compositions.

The cell therapy composition may be formulated and used in the form of oral dosage forms such as powders, granules, tablets, capsules, suspensions, emulsions, syrups and aerosols, external preparations, suppositories and sterile injection solutions, respectively, according to conventional methods. Examples of carriers, excipients and diluents that may be included include lactose, dextrose, sucrose, sorbitol, mannitol, xylitol, erythritol, maltitol, starch, gum acacia, alginate, gelatin, calcium phosphate, calcium silicate, cellulose, methylcellulose, microcrystalline cellulose, polyvinylpyrrolidone, water, methylhydroxybenzoate, propylhydroxybenzoate, magnesium stearate and mineral oil.

When formulating the cell therapy composition, it is prepared by using commonly used diluents or excipients such as fillers, extenders, binders, wetting agents, disintegrants and surfactants. Solid preparations for oral administration include tablets, pills, powders, granules, capsules and the like, and these solid preparations are prepared by mixing the above compound with at least one excipient, such as starch, calcium carbonate, sucrose or lactose, gelatin and the like. In addition to simple excipients, lubricants such as magnesium styrate talc are also used.

Liquid preparations for oral administration of the cell therapy composition include suspensions, oral solutions, emulsions and syrups. In addition to the commonly used simple diluents such as water and liquid paraffin, various excipients such as wetting agents, sweeteners, flavoring agents, preservatives and the like may be included.

Preparations for parenteral administration of the cell therapy composition include sterilized aqueous solutions, non-aqueous solutions, suspensions, emulsions, freeze-dried preparations and suppositories. Non-aqueous solvents and suspensions include propylene glycol, polyethylene glycol, vegetable oil such as olive oil, and injectable ester such as ethyl oleate. As a base for suppositories, Withepsol, Macrogol, Tween 61, cacao, laurin, glycerol gelatin and the like may be used.

In the present disclosure, the preferred dosage of the cell therapy composition varies depending on the patient's condition and weight, severity of disease, drug type, administration route and period, but it may be appropriately selected by a person skilled in the art. However, for desirable effects, the composition is preferably administered at 0.01 mg/kg to 10 g/kg per day, and preferably, at 1 mg/kg to 1 g/kg. Administration may be administered once a day, or may be administered in several divided doses.

The cell therapy composition may be administered to mammals such as rats, mice, livestock and humans through various routes. All methods of administration may be conventional methods, and for example, it may be administered through methods such as oral, rectal or intravenous administration.

In the present disclosure, the adhesion ability of neural stem cells cultivated on a substrate with controlled mechanical strength (e.g., hydrogel) may be characterized as being increased by 50% to 100%, but is not limited thereto.

In the present disclosure, the survival rate of neural stem cells cultivated on a substrate with controlled mechanical strength (e.g., hydrogel) may be characterized as being increased by 50% to 100%, but is not limited thereto. The survival rate may be calculated as a ratio of live cells/dead cells.

Hereinafter, the present disclosure will be described in more detail through examples. These examples are only for illustrating the present disclosure, and it is apparent to those skilled in the art that the scope of the present disclosure should not be construed as limited by these examples.

### Example 1: Method of Cultivating Neural Stem Cells in Hydrogel Matrix

The inventors of the present disclosure varied the mechanical strength of a hydrogel substrate purchased from Matrigen in the USA (Matrigen, LLC, 4790 Irvine Blvd STE 105-433, Irvine, CA 92620 https://matrigen.com/) in the range of 0.2 to 25 kPa, and an in vitro cultivation system was designed to cultivate NSC derived from embryonic spinal cord tissue on day 14 on the hydrogel substrate. Specifically, the spinal cord of a pregnant mouse embryo (14 days old) showing GFP fluorescence throughout the body was isolated, and neural stem cells were cultivated. The isolated neural stem cells were cultivated in the form of neurospheres and sub-cultivated in single cell units every week. For experiments related to the hydrogel matrix, cells in a neurosphere state (passages 2 to 5) were separated into single cells and cultivated on the hydrogel matrix for 24 hours.

### Example 2: Analysis Results of Changes in Cell Shape of Neural Stem Cells According to Increase in Mechanical Strength

Changes in the cell shape of neural stem cells due to an increase in mechanical strength were observed by fixing the neural stem cells with 4% paraformaldehyde 24 hours after cultivating the same on a hydrogel substrate, and after obtaining images of the cells using a confocal microscope, they were analyzed by using the ImageJ program.

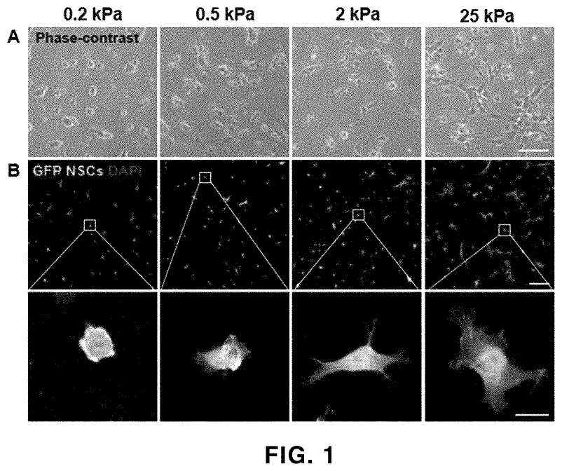

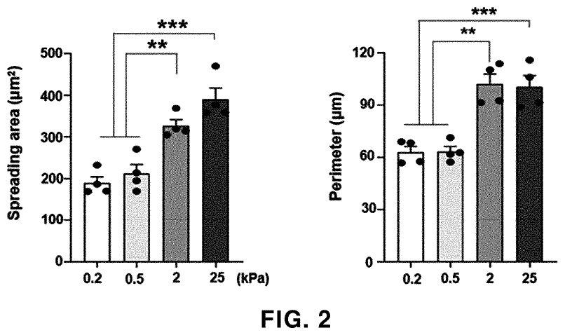

As a result, it was confirmed that as the mechanical strength increased, the spreading area and the attached cell perimeter effectively increased in terms of changes in cell shape (FIGS. 1 and 2).

### Example 3: Analysis Results of Survival Rate of Neural Stem Cells According to Increase in Mechanical Strength

The survival rate of neural stem cells according to an increase in mechanical strength was analyzed 24 hours after neurosphere-like cells (passages 2 to 4) were separated into single cells and the single nerve cells were cultivated on a hydrogel substrate. In order to observe the survival rate of cells, a live/dead cytotoxicity assay kit (Invitrogen, #L3224) was used as a reagent, showing Calcein AM (green, live cells) and Ethidium homodimer-1 (red, dead cells).

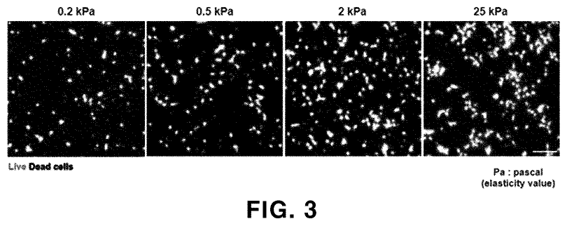

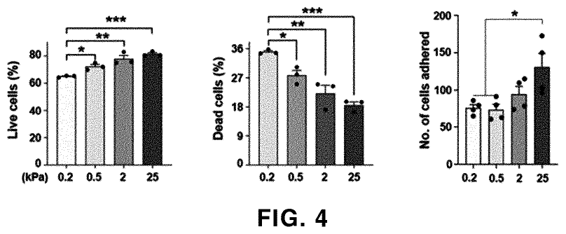

As a result, it was confirmed that as the mechanical strength increased, the proportion of live cells significantly increased, and the proportion of dead cells significantly decreased (FIGS. 3 and 4). Therefore, it indicates that the survival rate of neural stem cells increases as mechanical strength increases.

### Example 4: Analysis Results of Cell Receptors Responding to Mechanical Sensitivity

Cellular receptors that respond to mechanical sensitivity were identified using RT-PCR technique. Specifically, rat brain and lung tissues, which are known to express high levels of cell receptors that respond to mechanical sensitivity, were used as positive controls. After extracting RNA from neural stem cells cultivated on a general culture plate, cDNA was synthesized through reverse transcription, and PCR was performed.

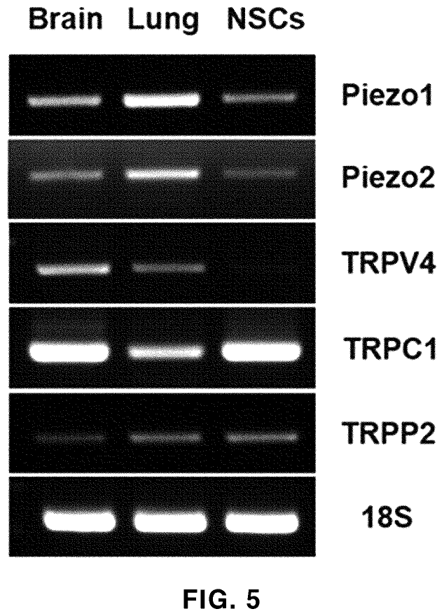

As a result, it was confirmed that cell receptors that respond to mechanical sensitivity, such as piezo1, piezo2, TRPC1 and TRPP2, were expressed in large quantities, and TRPV4 receptor was also expressed in small amounts (FIG. 5). This means that various mechanical sensitivity response receptors are expressed in spinal cord-derived neural stem cells.

### Example 5: Change in Calcium Ion Concentration Inside Cells According to Mechanical Sensitivity

The above mechanosensitive receptors are all well-known calcium ion channels. Therefore, in order to investigate the activation of mechanical sensitivity receptors by a hydrogel matrix, changes in intracellular calcium ions were analyzed. In order to analyze changes in calcium ion concentration inside cells through mechanical sensitivity receptors, single nerve cells were cultivated on 0.2 kPa and 25 kPa hydrogel substrates, and 24 hours later, the calcium level inside cells was measured using the fluo4AM calcium indicator. Calcium changes in neural stem cells according to mechanical sensitivity were confirmed in real time for 1 minute.

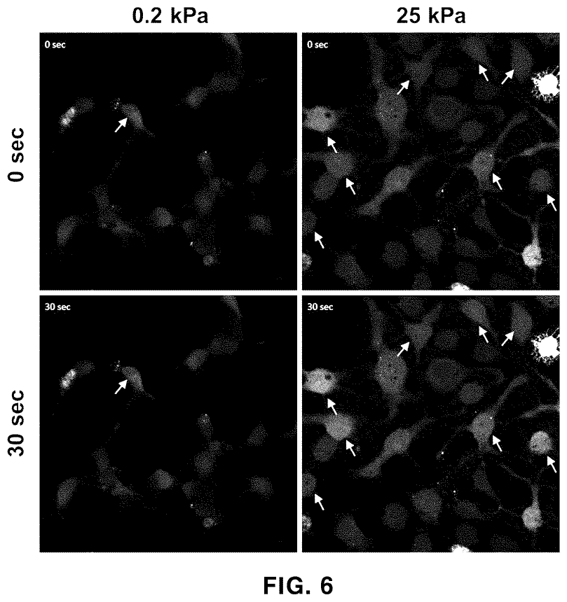

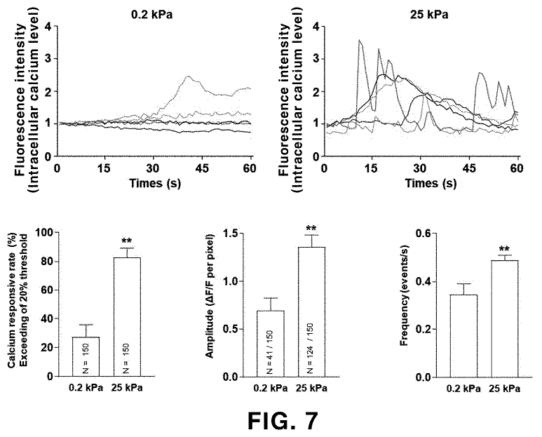

As a result, whereas the spontaneous calcium activity of neural stem cells was approximately 28% in a 0.2 kPa hydrogel matrix, it was confirmed that the spontaneous calcium level changed by approximately 80% in cells cultivated in a hard hydrogel matrix of 25 kPa. In addition, it was confirmed that the peak amplitude and frequency of spontaneous calcium levels in each cell showing calcium changes were significantly increased in the cell group cultivated on a hard substrate (FIGS. 6 and 7).

### Example 6: Analysis of Changes in Cell Shape and Cell Survival Rate by Cell Receptor Inhibitors in Response to Mechanical Sensitivity

In order to prove that the cell shape and cell survival rate due to the mechanical strength are changes due to mechanical sensitivity cell receptors, the results were confirmed by treating the cells with a cell receptor inhibitor that responds to mechanical sensitivity during cell culture.

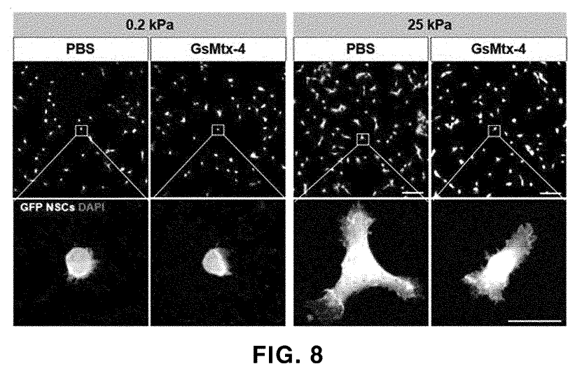

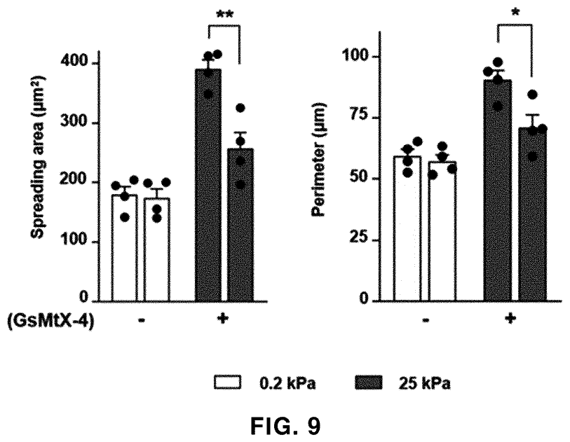

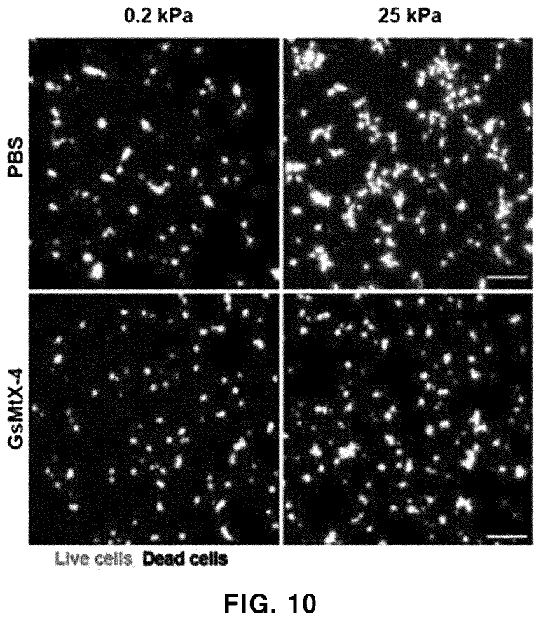

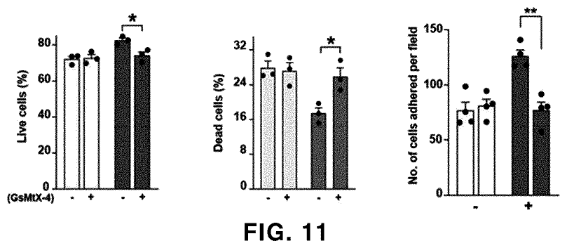

As a result, it was confirmed that cytoplasmic spreading was significantly reduced in the group where the cells were cultivated on a hard hydrogel substrate and treated with a mechanosensitive cell receptor inhibitor compared to the group that was not treated with the inhibitor (FIGS. 8 and 9). In addition, it was confirmed that the survival rate and adhesion ability of cells were significantly reduced in the group that was treated with the mechanosensitive cell receptor inhibitor (FIGS. 10 and 11). This suggests that cell adhesion ability and cell survival rate according to rigid mechanical sensitivity are achieved by mechanical sensitivity receptors.

### Example 7: Measurement of Changes in Cell Elasticity As the Mechanical Strength of the Substrate Increases

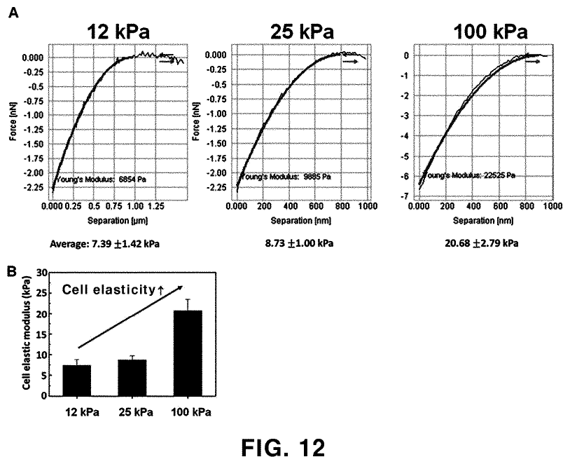

Changes in elastic modulus of neural stem cells were measured as the mechanical strength of the substrate increased (FIG. 12). The elasticity of cells was measured using atomic force microscopy (AFM). AFM is equipment that measures the elasticity of cells by using an optical system to measure the force that a cantilever (probe) exerts between the sample surface.

As a result, it was confirmed that the mechanical elasticity of the cell itself increased as the strength of the substrate increased (FIG. 12).

### Example 8: Role of Actin Fibers in Cell Elasticity due to Differences in Mechanical Strength

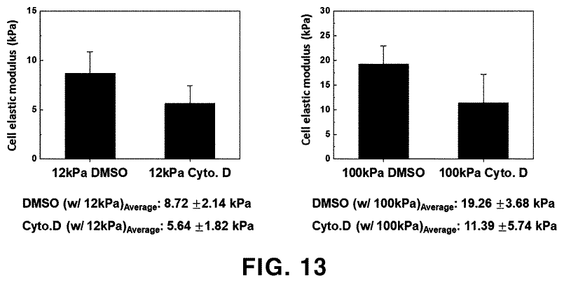

Changes in cell elasticity were measured to determine whether cell elasticity due to differences in the mechanical strength of the substrate was dependent on actin fibers (FIG. 13). The elasticity of cells was measured using atomic force microscopy (AFM). In order to determine whether the elasticity of cells itself changes when actin fibers are inhibited, cells were treated with actin fiber destabilizing drugs (Cytochalasin D, Cyto D), and then, the elasticity of cells was measured using AFM. Neural stem cells were cultivated on substrates with different mechanical strengths, and treated with an actin fiber destabilizing drug (Cyto. D), and the elasticity of the cells was measured to confirm the results (FIG. 13).

As a result, when they were treated with Cyto. D, the actin fibers of cells decreased, which resulted in a decrease in the elasticity of the cells themselves. Therefore, the results show that differences in the mechanical strength of the substrate affect the development of cell actin fibers, which plays an important role in the elasticity of the cell itself.

### Example 9: Confirmation of the Role of Actin Fibers for Cell Spontaneous Calcium Oscillation

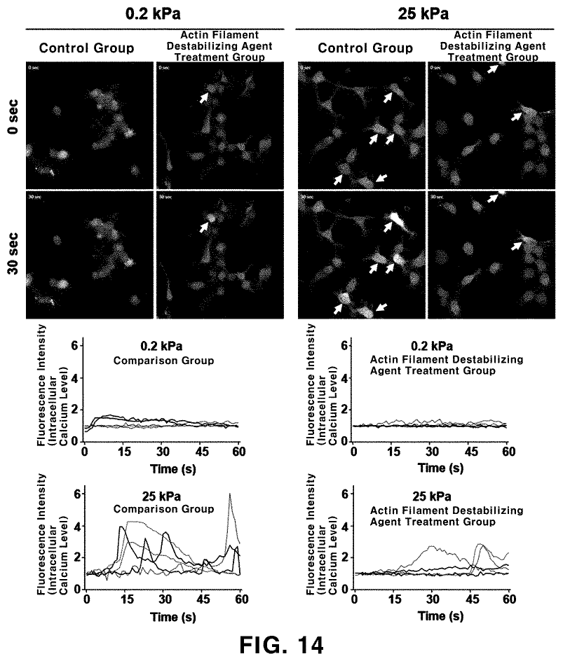

Spontaneous calcium oscillations were measured to determine whether spontaneous calcium oscillations due to differences in the mechanical strength of the substrate were dependent on actin fibers (FIG. 14). In this experiment, DMSO was treated with cytochalasin, which is an actin fiber destabilizing agent, or DMSO as a control group 30 minutes before the start of time-lapse microscopy imaging to measure calcium concentration, and images were acquired. In the left drawing of FIG. 14, cells in which spontaneous calcium oscillations were observed in time-lapse video recording (double O sec, 24 sec instantaneous photos shown) are indicated by arrows.

As a result, almost no spontaneous calcium oscillations were observed in cells cultivated on a 0.2 kPa substrate regardless of the treatment with the actin fiber destabilizing agent, but it can be observed that the number of cells exhibiting spontaneous calcium oscillations, which were clearly observed on 25 kPa substrates, was significantly reduced by treatment with actin filament destabilizing agents. The graph on the right drawing of FIG. 14 displays changes in intensity of the fluorescent signal representing the concentration of calcium during the 1-minute period of observing the cells, and it shows the increase in fluorescence signal intensity in cells cultivated on 25 kPa substrates disappeared due to actin filament destabilizers. It was confirmed that spontaneous calcium oscillations of neural stem cells in response to mechanical strength were reduced by treatment with cytochalasin D, which is an actin fiber destabilizing agent.

### Example 10: Confirmation of the Function of PIEZO1 Receptor in Cell Adhesion Ability and Cell Shape Change

The role of mechanical stimulation receptor piezol in cell adhesion and cytoplasmic expansion on stiff hydrogel substrates was determined. First of all, in order to investigate the function of Piezol, siRNA (small interfering RNA) was delivered to neural stem cells using electroporation, and Scramble (control group) and Piezo1 (experimental group, it temporarily reduces the gene expression rate of Piezo1) siRNA were used.

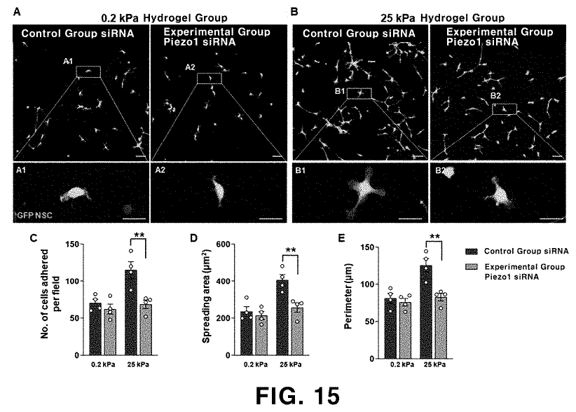

As a result, the cell adhesion ability and cytoplasmic expansion of the Piezo1 siRNA group cultivated on a 25 kPa hydrogel substrate were significantly reduced compared to the control group (scrambled siRNA, 25 kPa) (FIG. 15A to D).

1. A composition for cultivating stem cells, the composition comprising:
a substrate having a mechanical strength of 0.2 kPa to 500 kPa.

2. The composition of claim 1, wherein the substrate includes a hydrogel.

3. The composition of claim 2, wherein the hydrogel comprises a natural polymer, a synthetic polymer or a hybrid thereof.

4. The composition of claim 3, wherein the hydrogel further comprises an actin fiber activity regulator or a mechanoreceptor activity regulator.

5. The composition of claim 1, wherein the stem cells include neural stem cells.

6. A method for cultivating stem cells, the method comprising:
cultivating stem cells with the composition according to claim 1.

7. A scaffold material for cultivating stem cells, the scaffold material comprising:
a substrate having a mechanical strength of 0.2 kPa to 500 kPa.

8. The scaffold material of claim 7, wherein the substrate includes a hydrogel.

9. The scaffold material of claim 8, wherein the hydrogel comprises a natural polymer, a synthetic polymer or a hybrid thereof.

10. The scaffold material of claim 9, wherein the hydrogel further comprises an actin fiber activity regulator or a mechanoreceptor activity regulator.

11. The scaffold material of claim 7, wherein the stem cells include neural stem cells.

12. A cell therapy comprising the scaffold material according to claim 7 as an active ingredient.

13. The cell therapy of claim 12, wherein the cell therapy is for treating a nervous system disease.

14. A method for treating a nervous system disease, comprising administering to a subject in need thereof the cell therapy of claim 12.
